(19) United States
(12) Patent Application Publication
(10) Pub. No.: US 2026/0334784 A1
(43) Pub. Date: Oct. 29, 2026

(54) MOBILE DEVICE AND METHOD FOR DETERMINING AN EYE ORIENTATION OF THE EYES OF A PATIENT

(71) Applicant: Tooz Technologies GmbH, Aalen (DE)

(72) Inventors: David Dobbelstein, Ulm (DE); Doreen Wiedemann, Sömmerda (DE)

(73) Assignee: Tooz Technologies GmbH, Aalen (DE)

(21) Appl. No.: 19/464,677

(22) Filed: Jan. 30, 2026

Related U.S. Application Data
(63) Continuation of application No. PCT/EP2024/070610, filed on Jul. 19, 2024.

(30) Foreign Application Priority Data
Aug. 9, 2023 (DE) 10 2023 121 276.1

(51) Int. Cl. A61B 3/15; A61B 3/00

(52) U.S. Cl. CPC A61B 3/158; A61B 3/0083; A61B 3/0091

(57) ABSTRACT

A computer-implemented method for determining an eye alignment of eyes of a patient comprises outputting instructions to the patient, determining a position of the eyes relative to a mobile device; displaying a light visualization on a display unit of the modile device; capturing at least one image of the eyes with a camera of the mobile device; determining a position of a corneal reflection in each of the eyes of the patient based on the at least one captured image, and determining the eye alignment of both eyes using the position of the corneal reflection in each of the eyes of the patient.

### CROSS REFERENCE TO RELATED APPLICATIONS

This application is a continuation application of international patent application PCT/EP2024/070610, filed on Jul. 19, 2024, and designating the U.S., which claims priority to German patent application 10 2023 121 276.1 filed on Aug. 9, 2023, both of which are hereby incorporated by reference in their entireties.

### TECHNICAL FIELD

The disclosure is in the field of ophthalmology, optionally in the field of devices for assessing possible refractive errors of a patient. The disclosure may relate to a solution for determining an eye alignment of eyes of a patient. The disclosure may relate to a computer-implemented method to be performed by a mobile device having a display unit and a camera which is arranged on the same side of the mobile device as the display unit. The disclosure may also relate to a computer program, a computer-readable medium and a mobile device.

### BACKGROUND

Methods and apparatuses can be used by third parties to ascertain the presence and optionally the extent of strabismus, often referred as a squint, in a patient. Such examinations are often referred to as the Hirschberg test or Krimsky test. In conventional methods, an ophthalmologist carrying out the examination typically shines a penlight centrally at the patient, level with the nasal bridge. The light source is reflected off the cornea in both eyes and is visible there as a light spot. Since the fovea, which represents the region of sharpest vision, is typically not located centrally on the retina, the point of reflection is usually displaced slightly nasally in both eyes by an angle kappa. This displacement from the center is generally the same for healthy eyes. If strabismus (squint) is present in an eye, the point of reflection point is displaced horizontally and/or vertically. In the context of a medical examination, the extent of a possible strabismus is estimated by the physician on the basis of the manifestation of the angles kappa.

Devices can facilitate such an examination by the physician, wherein the devices can capture an image of the eyes and can subsequently determine a horizontal and/or vertical displacement of the points of reflection on the basis of the images. This can be implemented using dedicated devices which are provided specifically for this purpose. Alternatively, methods in which the physician captures an image of the eyes and ascertains any displacement of the points of reflection with the aid of a mobile device, such as a smartphone, may be used. For example, such a method is described in US 10,849,492 B2. In this context, a back-side camera of the mobile device, i.e. a camera arranged on the side of the mobile device opposite to the display, and an associated flash apparatus or a front-side camera with a separate, external light source may be used to capture images of points of reflection in the eyes of a patient. In this case, the points of reflection are created by a flash which may be provided by a flash function of the back-side camera of the mobile device. The images are recorded by a physician who can operate the mobile device from the side of the mobile device containing the display of the mobile device. A similar method is described in the following publication: Pundlik, S., et al. (2019): Development and preliminary evaluation of a smartphone app for measuring eye alignment. Translational vision science & technology, 8 (1), 19-19.

### SUMMARY

A computer-implemented method for determining an eye alignment of eyes of a patient is provided. The method is to be performed by a mobile device having a display unit and a camera arranged on a same side of the mobile device as the display unit. The method comprises outputting instructions to the patient that serve to assist the patient when aligning the mobile device relative to the eyes of the patient such that the display unit and the camera are positioned in a predetermined region relative to the eyes of the patient and face the eyes; determining a position of the eyes relative to the mobile device; displaying a light visualization on the display unit; capturing at least one image of the eyes with the camera such that a corneal reflection of the light visualization displayed on the display unit can be determined in each of the eyes of the patient in the captured image; and determining a position of the corneal reflection in each of the eyes of the patient based on the at least one captured image, and determining the eye alignment of both eyes using the position of the corneal reflection in each of the eyes of the patient.

### BRIEF DESCRIPTION OF THE DRAWINGS

In the drawings:

FIG. 1 shows a schematic illustration of a mobile device according to an optional implementation;

FIG. 2 shows a method for determining an eye alignment according to an optional implementation;

FIGS. 3A-3F show optional aspects of the method for determining an eye alignment according to an optional implementation.

### DESCRIPTION

In the following, details are set forth to provide a more thorough explanation of the disclosure. However, it will be apparent to those skilled in the art that these implementations may be practiced without these specific details. In other instances, well-known structures and devices are shown in block diagram form or in a schematic view rather than in detail to avoid obscuring the disclosure. In addition, features described hereinafter may be combined with each other, even if described with respect to different figures, unless specifically noted otherwise.

Equivalent or like elements or elements with equivalent or like functionality are denoted in the following description with equivalent or like reference numerals. As the same or functionally equivalent elements are given the equivalent or like reference numbers in the figures, a repeated description for elements provided with the equivalent or like reference numbers may be omitted. Hence, descriptions provided for elements having the equivalent or like reference numbers are mutually exchangeable.

Directional terminology, such as “top,” “bottom,” “below,” “above,” “front,” “behind,” “back,” “leading,” “trailing,” etc., may be used with reference to the orientation of the figures being described. Because parts of the disclosure, described herein, can be positioned in a number of different orientations, the directional terminology is used for purposes of illustration and is in no way limiting. It is to be understood that other implementations may be utilized, and structural or logical changes may be made without departing from the scope defined by the claims. The following detailed description, therefore, is not to be taken in a limiting sense.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Features from different implementations may be combined to form further implementations. For example, variations or modifications described with respect to one of the implementations may also be applicable to other implementations unless noted to the contrary.

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

One object of the disclosure may be to facilitate a determination of an eye alignment of eyes of a patient.

A computer-implemented method for determining an eye alignment of the eyes of a patient, to be performed or performed by a mobile device, is provided. In this case, the mobile device comprises a display unit and a camera, with the camera being arranged on the same side of the mobile device as the display unit. The method comprises outputting instructions to the patient that serve to assist the patient when aligning the mobile device relative to the eyes of the patient such that the display unit and the camera are positioned in a predetermined region relative to the eyes of the patient and face the eyes. Moreover, the method comprises determining a position of the eyes relative to the mobile device, displaying a light visualization on the display unit, and capturing at least one image of the eyes by means of the camera such that a corneal reflection of the light visualization displayed on the display unit can be determined in each of the eyes of the patient in the captured image. Further, the method comprises determining a position of the respective corneal reflection in the eye on the basis of the at least one captured image, and determining the eye alignment of both eyes using the position of the respective corneal reflection in the eye.

A computer program and/or a computer-readable medium is/are provided, which comprise instructions which, when executed by a mobile device having a display unit and a camera arranged on the side of the display unit, cause the mobile device to perform a method for determining an eye alignment for the eyes of a patient. In this case, the method comprises outputting instructions to the patient that serve to assist the patient when aligning the mobile device relative to the eyes of the patient such that the display unit and the camera are positioned in a predetermined region relative to the eyes of the patient and face the eyes. Moreover, the method comprises determining a position of the eyes relative to the mobile device, displaying a light visualization on the display unit, and capturing at least one image of the eyes by means of the camera such that a corneal reflection of the light visualization displayed on the display unit can be determined in each of the eyes of the patient in the captured image. Further, the method comprises determining a position of the respective corneal reflection in the eye on the basis of the at least one captured image, and determining the eye alignment of both eyes using the position of the respective corneal reflection in the eye.

A mobile device for determining an eye alignment of the eyes of a patient is proivded. The mobile device comprises a display unit and a camera, with the latter being arranged on the same side of the mobile device as the display unit. The mobile device is configured to output instructions to the patient in order to assist the patient when aligning the mobile device relative to the eyes of the patient such that the display unit and the camera are positioned in a predetermined region relative to the eyes of the patient and face the eyes. Moreover, the mobile device is configured to determine a position of the eyes relative to the mobile device, to display a light visualization on the display unit and to capture at least one image of the eyes by means of the camera such that a corneal reflection of the light visualization displayed on the display unit can be determined in each of the eyes of the patient in the captured image. Moreover, the mobile device is configured to determine a position of the respective corneal reflection in the eye on the basis of the at least one captured image and determine the eye alignment of both eyes using the position of the respective corneal reflection in the eye.

Determining the eye alignment may comprise determining a visual of the respective eye. Optionally, determining the eye alignment may comprise determining a deviation of the eye alignment between the two eyes of the patient. Optionally, determining the eye alignment may comprise determining a line of sight of the respective eye. In this case, the line of sight may denote an axis of the eye which runs through the fovea centralis of the eye through a nodal point of the eye to an object the eye is fixating on. Optionally, determining the eye alignment may comprise determining a deviation of the line of sight from a pupillary axis of the eye. In this context, the pupillary axis may denote an axis of the eye which runs through the apex of the cornea and the center of the pupil of the eye. In this case, the deviation between the line of sight and the pupillary axis may correspond to an angle conventionally referred to as angle kappa. The eye alignment may optionally be specified as an angle and/or in prism diopters.

A computer-implemented method can be defined as a method in which some or all method steps are executed by a computer, i.e. by an electronic data processing device. Optionally, all method steps may be executed by a computer. In this context, the computer may take the form of a mobile device. Here, a mobile device may be embodied as a mobile computer, optionally as a smartphone or as a tablet computer. A display unit may comprise a display or be embodied as such. Optionally, the display unit may be embodied as a touchscreen and accordingly be designed to enable user inputs. The mobile device may optionally comprise one or more processors and/or one or more data memories. Optionally, the mobile device may be designed to establish a communications link by way of one or more connection standards, for instance via GSM, WIFI, Bluetooth, NFC and/or via wired connection types such as USB, Ethernet, etc.

The camera being arranged on the same side of the mobile device as the display unit can mean that the camera faces the patient when the patient uses the mobile device while viewing the display unit. Optionally, the mobile device may comprise one or more (further) cameras that might be arranged on other sides of the mobile device. Optionally, the mobile device may have a front-side camera and a back-side camera, wherein the front-side camera may be arranged on the same side as the display unit and the back-side camera may be arranged on an opposite side of the mobile device to the display unit. In this case, the front-side camera may be embodied and arranged in such a way that the front-side camera is capable of capturing an image of the patient while the latter is looking at the display unit. In other words, the mobile device may allow the patient to record a „selfie“ by means of the front-side camera, i.e. a recording that the patient makes of themselves while viewing the display unit. In this case, the mobile device may be configured in such a way that the front side of the mobile device, on which the display unit and the front-side camera are formed, does not have a separate flash light source for providing a flash for recording images. By contrast, the back side may comprise such a separate flash light source; however, it is not suitable for recording images by means of the front-side camera on account of the emission region from the back side.

Outputting instructions to the patient may comprise outputting pieces of information and/or messages and/or commands by way of the display unit. For instance, it is possible to output a piece of information in relation to whether or not the mobile device is positioned in the predetermined region relative to the eyes. Optionally, the pieces of information may contain information in relation to the direction in which the mobile device should be moved and/or tilted in order to reach the predetermined region and/or improve the positioning and/or orientation of the mobile device. In addition to that or in an alternative, outputting instructions may optionally comprise outputting acoustic and/or haptic signals. This may allow the patient to receive the pieces of information even when the patient is not looking at the display unit. In this context, providing the patient with assistance may comprise user guidance, i.e. the user receives guidance continuously on the basis of the instructions so that the desired target is reached. Optionally, the mobile device may receive patient inputs by means of the display unit, for instance by actuating the touchscreen as a result of touching the display unit. As a result, the outputting of instructions and/or the performance of the method for determining the eye alignment may optionally be started and/or ended and/or paused.

The position of the eyes relative to the mobile device may optionally comprise an angular range in which the eyes extend relative to the camera or to any other element of the mobile device and/or relative to a visualization displayed by the display element. Optionally, the position may comprise a distance of the eyes from the mobile device. Optionally, provision may be made for the mobile device to be positioned at a distance of 20 cm to 70 cm and optionally at a distance of 30 cm to 50 cm in front of the eyes.

In this case, a light visualization may represent a light-emitting reproduction by the display element. Optionally, the light visualization may comprise a symbol and/or a pattern or be embodied as such. In this case, the light visualization may emit light in such a way that the light visualization generates a corneal reflection which can be captured by the at least one image and which can subsequently be used for the determination of the respective corneal reflection on the basis of the at least one image.

The disclosure offers the advantage that a patient is enabled to determine the eye alignment within the scope of a self-test using a commercially available mobile device. Optionally, this offers the advantage that the patient requires neither special hardware nor a further person for determining the eye alignment. Instead, the patient may perform the eye alignment determination by themselves and optionally use their own smartphone in the process. Consequently, the requirements in respect of the required hardware are low, and there is no need for staff, optionally no need for specially trained staff such as an ophthalmologist. Hence, the disclosure offers the advantage that, within the scope of a self-test, the patient is able to determine a possible deviation of the visual axis from a pupillary axis by themselves for each of the two eyes.

Further, the disclosure offers the advantage that, by outputting instructions to the patient, the latter can be assisted with performing the procedure, and hence the patient need not be assumed to have any special skills, knowledge or experience.

As a consequence, this offers the advantage that, with assistance by the patient, especially as regards the alignment of the mobile device relative to their eyes, an automated determination of the eye alignment may be performed. This may simplify the determination of the eye alignment and may be performed with only little effort by the patient should the need arise or as a matter of routine.

Further, the disclosure offers the advantage that such a determination of eye alignment can be implemented in mobile device applications. This may optionally be advantageous for those applications in which there is binocular virtual imaging for the patient or user, for instance when the mobile device is used for augmented reality applications. In this case, binocular virtual imaging may represent an application in which different regions of the display unit are viewed in each case by only one of the two eyes of the patient, and the patient or user experiences a three-dimensional visual experience as a result of a targeted output of visual content in the different regions of the display unit. In this case, optionally, planned binocular virtual imaging for the patient may be adapted taking into account the eye alignment in order to improve the visual experience for the patient. This may optionally be advantageous if the patient suffers from heterophoria(s) or heterotopia(s) (strabismus). The angle kappa can be used to assess and optionally measure the stereoscopic vision. Optionally, it is possible to provide an estimate regarding the manifestation and/or quality of the spatial, three-dimensional vision of the user. Optionally, the determined eye alignment may be used to output a recommendation as to whether or not viewing a binocular virtual imaging is recommended to the patient. Optionally, the mobile device may also be used to determine the eye alignment of the patient for the purpose of providing, and optionally using said determined eye alignment to adapt, a binocular virtual imaging on a different device.

Optionally, the disclosure offers the advantage that it may allow simple recognition and/or determination of strabismus, and the choice of a suitable type and/or manifestation of spectacle lenses and/or other visual aids for the patient may be facilitated and/or improved thereby.

Moreover, the disclosure offers the advantage that a back-side camera of a mobile device and an associated flash light source are not needed for this method, and this represents an additional innovation over and delimitation from the method from US 10,849,492 B2 and the prior art. The method described in US 10,849,492 B2 requires a flash light source to generate a point of reflection. However, since flash light sources in conventional mobile devices are usually installed only with the back-side camera, and hence the camera facing away from the patient, independent implementation by the patient is made much more difficult, and implementation with the front-side camera requires an additional light source. The method according to the disclosure offers the advantage that it allows the method to be implemented independently by the patient without the requirement of an additional light source or flash light source. Instead, the corneal reflection may be provided by a light visualization displayed on the display unit. A perspective offset between camera, eye and light visualization that may arise therefrom can be corrected relatively by measuring the eye position.

Determining the position of the respective corneal reflection in the eye may comprise determining a relative position of the corneal reflection with respect to a pupil center and/or an iris center of the respective eye. This may offer the advantage that a viewing direction of the eye can be ascertained or estimated. Determining the eye alignment of both eyes may comprise determining a deviation of a line of sight from a pupillary axis for each of the two eyes. Optionally, the deviations for the two eyes may be compared with each other and used to ascertain whether or not the patient presents a strabismus and optionally how severe the strabismus is.

The light visualization displayed on the display unit may have such a brightness and/or size on the display unit that the light visualization can be determined by performing an image evaluation of the at least one image by way of the mobile device. In this case, the image evaluation may optionally be performed by the mobile device itself, i.e. using one or more processors of the mobile device. In an alternative to that or in addition, the image evaluation may be implemented on a server remote from the mobile device. To this end, the mobile device may transmit the captured image to the server via a network connection and receive a result of the image evaluation from the server.

The method may optionally further include displaying a fixation visualization on the display unit before and/or while displaying the light visualization and capturing the at least one image, in order to direct a gaze of the patient onto the fixation visualization. This may offer the advantage that the eyes can adopt a predetermined viewing direction when the image is captured, and so correspondingly predetermined conditions can be created for determining the position of the respective corneal reflection.

Determining the position of the eyes relative to the mobile device may comprise a determination, optionally a three-dimensional determination, of a position, optionally a spatial position, of the eyes relative to the camera and/or relative to the fixation visualization. This may offer the advantage that a distance and/or positioning of the patient relative to the mobile device can be reliably detected during the method, and a perspective offset of the light visualization relative to the eyes and/or relative to the camera can be corrected, and any potential parallax can be taken into account, and an accuracy of the determination of the eye alignment can be improved thereby.

The instructions may be output to the patient by means of the display unit. Optionally, outputting the instructions may comprise a graphical user interaction. This offers the advantage that no additional hardware is required for outputting the instructions. Moreover, this may offer the advantage that the patient already directs their gaze onto the display unit for the purpose of observing the instructions on the display unit, said display unit then being used to also display the light visualization given a correct alignment of the mobile device relative to the eyes. This may hence promote efficient performance of the method.

In this case, the display unit and the camera are optionally arranged on that side of the mobile device which faces the eyes of the patient when said mobile device is used by the patient. The display unit may be embodied as a display and optionally as a touchscreen. Moreover, the mobile device may be configured to allow the determination of the eye alignment of the eyes of the patient within the scope of a self-test by the patient.

As a result of the light visualization being displayed on the display unit, the display unit can be used as a light source for generating the corneal reflection. This may offer the advantage that no further separate light source need be provided. Moreover, this may offer the advantage that the light visualization displayed on the display unit is adaptable in terms of its position on the display unit, the brightness and the size.

Optionally, the light visualization may comprise a symbol and/or a pattern or be embodied as such. Optionally, the symbol and/or pattern may be configured such that the corneal reflection thereof is reliably identifiable in the at least one captured image and optionally reliably distinguishable from any other corneal reflection. Optionally, the symbol may comprise one or more of the following geometric shapes or be embodied as such: a point, a circle, an ellipse, a polygon, a triangle, a quadrilateral, a square, a rectangle, a trapezoid, a rhombus and a cushion shape. Optionally, the pattern may comprise one or more of the following designs or be embodied as such: a grid, a ring structure, a meander, a star, a trademark, a letter, a numeral and a symbolic figure. These examples are merely intended to indicate exemplary configurations of the light visualization and should not be construed as limiting. The use of other forms for symbols and patterns is possible.

Optionally, with respect to the surroundings on the display unit, the light visualization may have an optical brightness contrast within a predetermined range. Optionally, the light visualization may be displayed with such brightness that, relative to the surroundings of the lighting means visualization, the light visualization has a brightness contrast on the display unit of at least 3:1, optionally at least 5:1, optionally at least 10:1, optionally at least 20:1, optionally at least 50:1, optionally at least 100:1 and optionally at least 1000:1.

Optionally, the display unit may be at least partially or completely darkened outside of the light visualization while the light visualization is displayed and/or the at least one image is captured. This may bring about a further increase in the brightness contrast and offer the advantage that a determination of the position of the corneal reflection of the light visualization can be simplified further.

Optionally, the light visualization may be displayed on the display unit in such a way that the light visualization has a pronounced color contrast relative to the surroundings of the light visualization on the display unit. Thus, the light visualization may be presented in one or more colors which are complementary to colors of any other elements displayed on the display unit. Thus, the light visualization may optionally be displayed in white while surrounding elements on the display unit are displayed in black. Thus, the light visualization may optionally be displayed in green or blue while surrounding elements on the display unit are displayed in red, or vice versa. These examples should merely explain the use of a color contrast and should not be construed as limiting. Other color combinations are possible.

The features and implementations mentioned above and explained below should not only be considered to be disclosed in the respective explicitly mentioned combinations in this case but are also encompassed by the disclosure in other technically advantageous combinations and implementations.

All the disclosures described for the method should also to be regarded as being disclosed for the computer program and the mobile device, and vice versa.

The same or similar elements in the various implementations are denoted by the same reference symbols in the following Figs. for reasons of simplicity.

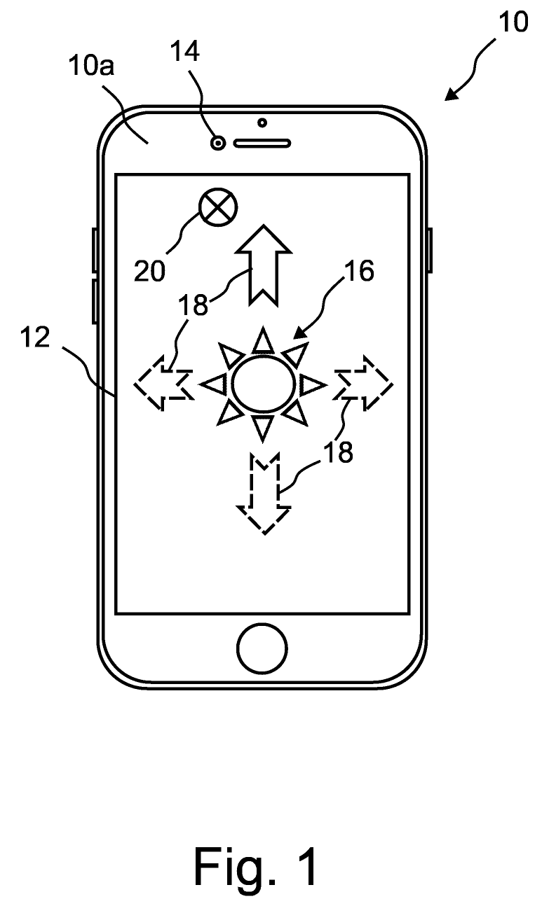

FIG. 1 shows a schematic illustration of a mobile device 10 for determining an eye alignment of the eyes of a patient according to an optional implementation. The mobile device 10 comprises a display unit 12 and a camera 14 which is arranged on the same side of the mobile device 10 as the display unit 12. In this case, the display unit 12 and the camera 14 may be arranged on a side of the mobile device 10 that faces the eyes of the patient when said mobile device 10 is used by the patient. This may be the front side 10a of the mobile device.

The mobile device is configured to perform the following steps:
(i) outputting instructions to the patient that serve to assist the patient when aligning the mobile device 10 relative to the eyes of the patient such that the display unit 12 and the camera 14 are positioned in a predetermined region relative to the eyes of the patient and face the eyes.
(ii) determining a position of the eyes relative to the mobile device 10.
(iii) displaying a light visualization 16 on the display unit 12.
(iv) capturing at least one image of the eyes by means of the camera 14 such that a corneal reflection of the light visualization 16 displayed on the display unit 12 can be determined in each of the eyes of the patient in the captured image.
(v) determining a position of the respective corneal reflection in the eye on the basis of the at least one captured image, and determining the eye alignment of both eyes using the position of the respective corneal reflection in the eye.

The mobile device 10 may be embodied as a smartphone or as a tablet computer in this case. The display unit 12 may be embodied as a display and optionally as a touchscreen. Optionally, the mobile device 10 may be configured to allow the determination of the eye alignment of the eyes of the patient within the scope of a self-test by the patient.

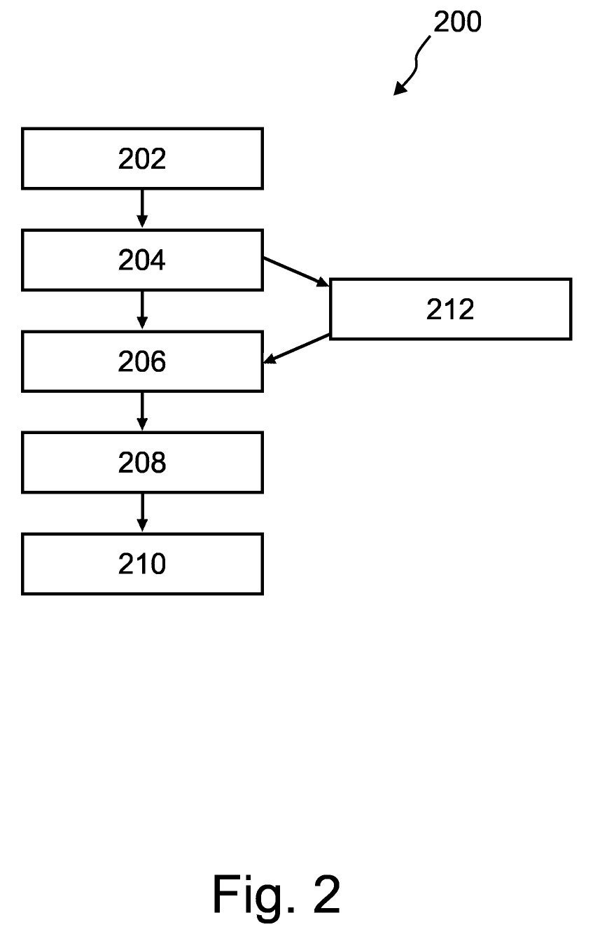

The mobile device 10 may be configured to perform the computer-implemented method, described below with reference to FIG. 2, according to an optional implementation.

FIG. 2 schematically shows a computer-implemented method 200 for determining an eye alignment of the eyes of a patient, to be performed by a mobile device 10 having a display unit 12 and a camera 14, which is arranged on the same side of the mobile device 10 on which the display unit 12 is also arranged.

In a step 202, the method 200 comprises outputting instructions 18 to the patient that serve to assist the patient when aligning the mobile device 10 relative to the eyes of the patient such that the display unit 12 and the camera 14 are positioned in a predetermined region relative to the eyes of the patient and face the eyes in the process. Outputting 202 the instructions to the patient may optionally be implemented by means of the display unit 12 and optionally comprise a graphical user interaction. Outputting instructions may optionally comprise the display of symbols and/or textual content to the patient in order to instruct the patient to change or maintain a positioning of the mobile device relative to the eyes of the patient. For example, such symbols, as illustrated, may comprise one or more arrows which indicate a direction in which the patient should move the mobile device in order to achieve the intended positioning. The selection and/or type and/or size and/or color of the displayed symbols may change over the course of the procedure of aligning the mobile device 10 and may adapt to the new positioning of the mobile device during and/or after a movement by the patient. For example, it might be necessary to move the mobile device upward according to the displayed symbols of the instructions 18, as may be indicated by a corresponding upward-directed arrow. The fact that the other arrows are represented by dashed lines may mean that these are not displayed in the present positioning of the mobile device 10 relative to the eyes.

In a step 204, the method comprises determining a position of the eyes relative to the mobile device and/or relative to the light visualization. Determining 204 the eye alignment of both eyes may comprise determining a deviation of a line of sight from a pupillary axis for each of the two eyes. Steps 202 and 204 may optionally be performed at least partially in parallel, i.e. with a temporal overlap.

In a step 206, the method 200 comprises displaying a light visualization 16 on the display unit 12. The light visualization 16 may have such a brightness and/or size on the display unit 12 that the light visualization 16 can be determined by performing an image evaluation of the at least one image by way of the mobile device 10.

In a step 208, the method 200 comprises capturing at least one image of the eyes by means of the camera 14 such that a corneal reflection of the light visualization 16 can be determined in each of the eyes of the patient in the captured image.

In a step 210, the method 200 comprises determining a position of the respective corneal reflection in the eye on the basis of the at least one captured image, and determining the eye alignment of both eyes using the position of the respective corneal reflection in the eye. Determining 210 the position of the respective corneal reflection in the eye may comprise determining a relative position of the corneal reflection with respect to a pupil center and/or an iris center of the respective eye.

Optionally, the method 200 may further comprise displaying 212 a fixation visualization 20 on the display unit 12 before and/or while displaying 206 the light visualization 16 and capturing 208 the at least one image, in order to direct a gaze of the patient onto the fixation visualization 20. The fixation visualization may optionally comprise the display of a stimulus point, which may optionally be displayed in flickering fashion. Optionally, step 212 may further comprise capturing and evaluating one or more images in order to ascertain whether or not the patient is directing their gaze at the fixation visualization. Should this not be the case, step 212 may optionally be repeated one or more times. The fixation visualization 20 may optionally be displayed centrally on the display unit 12 between the camera 14 and the light visualization 16. The position at which the fixation visualization should be displayed may optionally be ascertained on the basis of information about the properties of the display unit 12 and/or the position of the camera in the mobile device 10. Optionally, the information to be used for ascertaining the position at which the fixation visualization should be displayed may be provided in advance, for instance in a provided data record.

Determining 204 the position of the eyes relative to the mobile device 10 may comprise a determination of a position of the eyes relative to the camera 14 and/or relative to the fixation visualization 20.

The method 200 may be configured in such a way that the method allows the patient to perform a self-test.

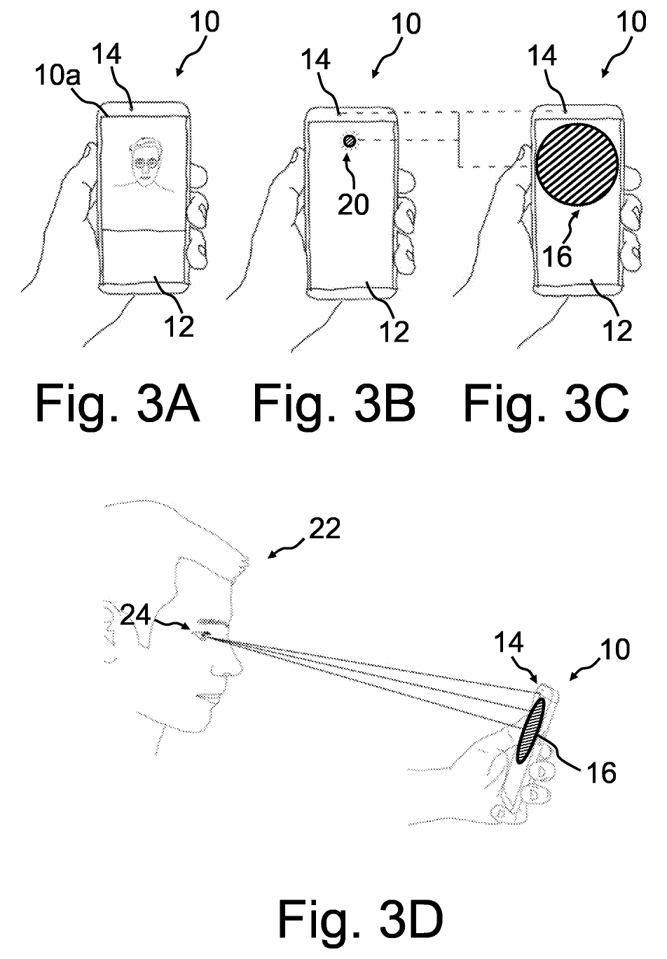

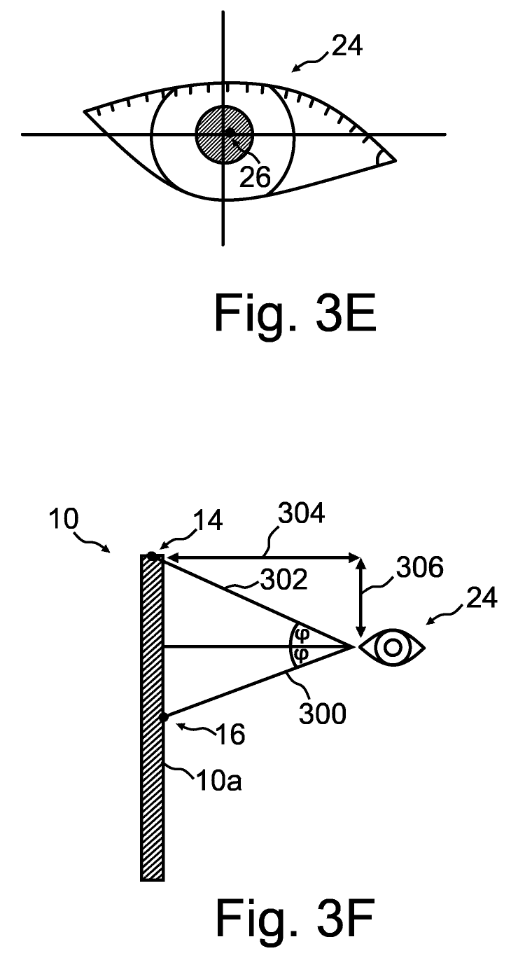

Further optional aspects are explained below on the basis of FIGS. 3A-3F.

FIGS. 3A and 3B show a mobile device 10 according to an optional implementation from the view of the patient when determining an eye alignment by means of the mobile device 10.

In this case, FIG. 3A represents an optional visualization of the captured camera image during the determination of the position of the eyes relative to the mobile device. This may allow the patient to check the captured eye positions in the captured image, in order to capture a new image or terminate the process in the event of an incorrect identification of the eyes.

FIG. 3B shows, by way of example, the display of a fixation visualization 20 on the display unit 12, the purpose of which is to direct a gaze of the patient onto the fixation visualization.

FIG. 3C shows, by way of example, the display of a light visualization 16 on the display unit 12 during the capture of at least one image of the eyes by means of the camera 14. As a result, a corneal reflection of the light visualization in the captured image may be determined in each eye. In this case, the fixation visualization 20 may be displayed directly before and/or during the display of the light visualization 16 in order to avoid the patient averting their gaze from the fixation visualization. As represented by dashed lines, the camera 14, the fixation visualization 20 and the light visualization 16 may have a predetermined arrangement with respect to each other in this case, and this may be used in the determination of the position of the respective corneal reflection.

The light visualization may be displayed briefly immediately after the display of the fixation visualization, for example 100 ms to 500 ms thereafter, and then one or more images may be captured immediately by the camera. Depending on the quality, a video frame may also be used for this purpose; however, the assumption is made that a photograph is able to provide a higher image quality than a video frame. What a very rapid display of the light visualization 16 achieves is that the fixation point of the eye is still aligned by the fixation visualization and not yet directed at the pattern now visible on the display unit.

FIG. 3D shows a schematic side view of a relative positioning of the mobile device 10 with respect to the eyes that may be suitable for determining the eye alignment. In this case, the lines visualize the direction vectors from the eye to the light visualization 16, to the fixation visualization 209 and to the camera 14. These may be included in the determination of the respective eye alignment in order to take any parallax error into account.

FIG. 3E shows, by way of example, an image of an eye 24 of the patient 22 in which a corneal reflection 26 of the light visualization 16 is identifiable. This may optionally be gathered from the captured image. It is evident here that the light visualization 16 is not located on the pupil center and/or iris center indicated by the crossed lines but has a horizontal and vertical offset from said pupil center and/or iris center.

This may be used as a basis for determining a vertical and/or horizontal displacement of a line of sight of the eye from a pupillary axis of the eye. This may optionally be calculated by the mobile device 10.

The schematically illustrated distances and direction vectors, which are plotted in FIG. 3D, are explained in more detail in FIG. 3F, in exemplary and schematic fashion. In this case, the line 300 indicates the connection vector between a center or another marked point of the light visualization and the eye 24, for example the pupil center and/or iris center, and 302 indicates the connection vector between the camera 14 and the eye 24. Arrow 304 indicates the distance of the eye 24 from the front side 10a of the mobile device 10 in the z-direction, and arrow 306 indicates the distance of the eye from the camera 14 in the y-direction. Optionally, a further distance in the x-direction between the camera 14 and the eye 24 may be determined. In this case, provision may be made for the eyes to be arranged centrally with respect to the camera, i.e. a distance of the nasal bridge from the camera is approximately zero in the x-direction. The consequence thereof may be that the x-distance of one of the eyes is approximately between 3.15 cm and -3.15 cm.

Optionally, the patient may be encouraged to position and align the mobile device at a predetermined position such that the distances from the eye 24 in the y-direction to the camera 14 and to the center of the light visualization 16 are approximately the same. To this end, the y-distance of the eye from the camera may optionally be approximately 2 cm. The predetermined position of the eye may optionally be at a distance of approximately 40 cm from the front side of the mobile device.

To determine the fixation direction of the eye 24, i.e. the line of sight, the position of the respective eye in relation to the mobile device is relevant. In this case, a three-dimensional position of the eye relative to the mobile device may optionally be calculated as follows:
Variant A) On the basis of a depth sensor installed in the mobile device, a depth map is created and optionally adapted in terms of perspective to the image section of the camera 14. A position of the individual eye may be captured in a two-dimensional projection in the captured image with the aid of camera-based facial recognition (algorithmic solution). On the basis of the identified two-dimensional position in the captured image, a distance of the eye in the z-direction may be gathered from the depth map (=> z-component in the 3D position). On the basis of an intrinsic calibration of the camera and the two-dimensional position of the individual eye in the camera image, it is possible to calculate x- and y-components of the three-dimensional position of the individual eye.
A variant B) described below is similar in many aspects to variant A, but alternatively the x- and y-position components are also taken from the depth map. In this case, it is possible to take account of whether a depth map has been distorted in perspective by an adaptation to the image section of the front camera. This may be corrected where necessary.
Further, a variant C) is described below as an alternative without a depth sensor. In this case, the z-distance is calculated from the captured camera image. In this case, the calibration may require a known object size, which must be located at a similar distance as the eye in the camera image section at least once (e.g. a credit card may be held in front of the eye or on the forehead). In this way, the current z-distance can be determined from the detected size of the object. The identified distance may in turn be used to determine the pupil size and/or the iris size of the individual eye in the camera image. This may subsequently be used as a reference parameter for determining the distance in the z-direction. The x- and y-components of the position may be determined in accordance with variant A.
Furthermore, a variant D) is described below as an alternative without a depth sensor and without a reference object. In this case, the z-distance is calculated from the captured camera image, and an iris size and/or pupil size is used as a reference parameter for determining the distance in the z-direction. x- and y-components of the position may be determined in accordance with variant A.

The position of the eyes may optionally be determined regularly at defined time intervals. Optionally, the position is determined immediately before the light visualization is displayed. Optionally, the eye position may also be determined while the instructions are output to the patient for providing assistance with the alignment of the mobile device, in order to always obtain accurate and updated knowledge of any deviations from the positioning of the mobile device in the predetermined region relative to the eyes.

As soon as the eyes are in the predetermined three-dimensional position relative to the mobile device or as soon as the mobile device is in the predetermined region relative to the eye – threshold values as regards the three-dimensional position (x, y, z) are optionally predeterminable in this respect – it is possible to additionally direct the fixation direction of both eyes.

A perspective offset between the light visualization on the display unit and the camera with respect to the eye can be compensated by virtue of the patient fixating their gaze centrally between the camera and the light visualization. This is optionally achieved by directing the viewing direction using a fixation visualization, for instance in the form of a flashing visual stimulus. The stimulus optionally flashes quickly for a defined period of time (e.g. for 2 s). It is possible to proceed automatically directly thereafter.

The position of the flashing stimulus is optionally calculated from the position of the camera, the position of the light visualization on the display unit and the position of the eye to be measured. Optionally, these form an isosceles triangle (see FIG. 3F), in which the flashing stimulus is located centrally between the camera and pattern, level with the two eyes.

The corneal reflection of the light visualization of the respective eye may be determined in relation to the pupil center and/or iris center, for instance by means of algorithmic image processing, and it is possible to determine a horizontal and/or vertical deviation between the corneal reflection and the pupil center and/or iris center. The deviation may optionally be specified in mm and/or in prism diopters. To this end, it is now possible to calculate the angle kappa, which specifies the difference between the optical axis and the line of sight.

The corneal reflection is usually displaced slightly nasally in the horizontal direction on both eyes by the angle kappa. This offset may be taken into account when determining the horizontal/vertical displacement of the line of sight, wherein the assumption is optionally made that there is no strabismus in at least one eye.

The line of sight may optionally be corrected in perspective by a known perspective offset from the known three-dimensional relationship from the respective eye, the respective light visualization and the respective camera.

### LIST OF REFERENCE SYMBOLS

10 Mobile device
10a Front side of the mobile device
12 Display unit
14 Camera
16 Light visualization
18 Instruction
20 Fixation visualization
22 Patient
24 Eye of the patient
26 Corneal reflection
200 Method for determining an eye alignment
202-212 Method steps
300 Connection vector between the center of the light visualization and the eye
302 Connection vector between the camera and the eye
304 z-distance between front side of the mobile device and the eye
306 y-distance between the camera and the eye

What is claimed is:

1. A computer-implemented method for determining an eye alignment of eyes of a patient, to be performed by a mobile device having a display unit and a camera arranged on a same side of the mobile device as the display unit, the method comprising:
outputting instructions to the patient that serve to assist the patient when aligning the mobile device relative to the eyes of the patient such that the display unit and the camera are positioned in a predetermined region relative to the eyes of the patient and face the eyes;
determining a position of the eyes relative to the mobile device;
displaying a light visualization on the display unit;
capturing at least one image of the eyes with the camera such that a corneal reflection of the light visualization displayed on the display unit can be determined in each of the eyes of the patient in the captured image; and
determining a position of the corneal reflection in each of the eyes of the patient based on the at least one captured image, and determining the eye alignment of both eyes using the position of the corneal reflection in each of the eyes of the patient.

2. The method as claimed in claim 1, wherein determining the position of the corneal reflection in each of the eyes of the patient includes determining a relative position of the corneal reflection with respect to a pupil center and/or an iris center of a respective eye of the eyes of the patient.

3. The method as claimed in claim 1, wherein determining the eye alignment of both eyes includes determining a deviation of a line of sight from a pupillary axis for each of the two eyes.

4. The method as claimed in claim 1, wherein the light visualization has such a brightness and/or size on the display unit that the light visualization can be determined by performing an image evaluation of the at least one image by way of the mobile device.

5. The method as claimed in claim 1, further comprising displaying a fixation visualization on the display unit before and/or while displaying the light visualization and capturing the at least one image to direct a gaze of the patient onto the fixation visualization.

6. The method as claimed in claim 5, wherein determining the position of the eyes relative to the mobile device includes determining a position of the eyes relative to the camera and/or relative to the fixation visualization.

7. The method as claimed in claim 1, wherein the instructions are output to the patient via the display unit.

8. The method as claimed in claim 1, wherein outputting instructions to the patient includes a graphical user interaction.

9. The method as claimed in claim 1, wherein the method is configured in such a way that the method allows a self-test to be performed by the patient.

10. A non-transitory computer-readable medium, comprising instructions which, when executed by a mobile device having a display unit and a camera arranged on a same side of the mobile device as the display unit, cause the mobile device to perform a method for determining an eye alignment of eyes of a patient, the method including:
outputting instructions to the patient that serve to assist the patient when aligning the mobile device relative to the eyes of the patient such that the display unit and the camera are positioned in a predetermined region relative to the eyes of the patient and face the eyes;
determining a position of the eyes relative to the mobile device;
displaying a light visualization on the display unit;
capturing at least one image of the eyes with the camera such that a corneal reflection of the light visualization displayed on the display unit can be determined in each of the eyes of the patient in the captured image; and
determining a position of the corneal reflection in each of the eyes of the patient based on the at least one captured image, and determining the eye alignment of both eyes using the position of the corneal reflection in each of the eyes of the patient.

11. A mobile device for determining an eye alignment of eyes of a patient, the mobile device comprising:
a display unit; and
a camera arranged on a same side of the mobile device as the display unit;
wherein the mobile device is configured to:
output instructions to the patient that serve to assist the patient when aligning the mobile device relative to the eyes of the patient such that the display unit and the camera are positioned in a predetermined region relative to the eyes of the patient and face the eyes of the patient;
determine a position of the eyes of the patient relative to the mobile device;
display a light visualization on the display unit;
capture at least one image of the eyes with the camera such that a corneal reflection of the light visualization displayed on the display unit can be determined in each of the eyes of the patient in the captured at least one image; and
determine a position of the corneal reflection in each of the eyes of the patient based on the at least one captured image, and determine the eye alignment of both eyes using the position of the corneal reflection in each of the eyes of the patient.

12. The mobile device as claimed in claim 11, wherein the mobile device is a smartphone or a tablet computer.

13. The mobile device as claimed in claim 11, wherein the side of the mobile device on which the display unit and the camera are arranged faces the eyes of the patient when said mobile device is used by the patient.

14. The mobile device as claimed in claim 11, wherein the display unit includes at least one of a display and a touchscreen.

15. The mobile device as claimed in claim 11, wherein the mobile device is configured to allow the determination of the eye alignment of the eyes of the patient within a scope of a self-test by the patient.
